(19) United States
(12) Patent Application Publication
(10) Pub. No.: US 2003/0003104 A1
(43) Pub. Date: Jan. 2, 2003

(54) Method for removal of pathogens from a localized environment

(76) Inventors: John Mottola, Maniboba (CA); John Hare, Manitoba (CA)

Correspondence Address: Michael R. Williams, Ade & Company, 1700-360 Main Street, Winnipeg Manitoba, R3C 3Z3, CA

(21) Appl. No.: 10/183,549

(22) Filed: Jun. 28, 2002

Related U.S. Application Data
(60) Provisional application No. 60/301,825, filed on Jul. 2, 2001.

(51) Int. Cl. A61K 39/40

(52) U.S. Cl. 424/164.1

(57) ABSTRACT

A method of purging enterotoxigenic organisms from an environment is described whereby contamination of other animals by the organism is prevented.

### PRIOR APPLICATION

This application claims priority under 35 USC §119(e) to U.S. Ser. No. 60/301,825, filed Jul. 2, 2001.

### FIELD OF THE INVENTION

The present invention relates generally to the field of passive immunity.

### BACKGROUND OF THE INVENTION

Bacterial strains of Escherichia coli are categorized by their surface antigens; O and H antigen serotypes are one means of classification. E. coli strain O157:H7, a pathogen first seen in 1982 during an outbreak of bloody diarrhea (hemorrhagic colitis) that was linked to undercooked hamburger, appears to harmlessly live in the gut of cattle, just as a strain of Salmonella (also pathogenic to humans) resides harmlessly in the ovaries of chickens. The trouble arises when these bacteria move from their natural hosts to the bodies of humans. From more than 5,200,000 total estimated bacterial cases of foodborne illness per year in the United States, about 73,000 are due to E. coli O157:H7, and about 1,400,000 are due to nontyphoidal Salmonella. This bacterium may not only be present in meat and dairy products, but may be found in any food—animal or vegetable—that has accidentally come in contact with cattle intestines or their contents. Infected humans also may spread the bacteria to uninfected individuals. It is believed that from two to 10 living E. coli O157:H7 bacteria are all that are necessary for a productive infection.

In addition to several widely reported cases of tainted hamburger and other food products causing death or illness in North America, during May 2000, in Walkerton, Ontario, 7 people died from drinking water contaminated with E. coli while hundreds of others suffered from symptoms of the disease.

Campylobacter jejuni is a major human enteropathogen and is implicated in more than 95% of the cases of campylobacteriosis in the United States (Karmali et al., 1983, J Infect Dis 147: 243-246). Species of Campylobacter are non-pathogenic in some animals, for example, chickens, but can be transmitted to humans.

Clostridium difficile is responsible for colitis in humans, antibiotic associated diarrhea and virtually all cases of pseudomembranous colitis. This bacterium is often a problem during antibiotic treatments. Specifically, the antibiotics kill other intestinal flora and allow C. difficile to over grow, thereby causing diarrhea. The disease develops as a result of the production of two large toxins: toxin A and toxin B.

U.S. Pat. No. 4,748,018 teaches a method of passively immunizing a mammal against an antigen by feeding said mammal material derived from eggs so that tolerance to the egg material is developed and then feeding said mammal anti-antigen antibodies obtained from the eggs of a hen immunized by the antigen.

U.S. Pat. No. 5,080,895 teaches a method of preventing or treating an intestinal infectious disease in a neonatal mammal comprising feeding to the neonatal mammal egg material from an immunized hen.

U.S. Pat. No. 5,837,825 teaches a fusion protein comprising the B subunit of the labile toxin (LT-B) of E. coli and part of the flagellin protein of C. jejuni which is partially purified and used to vaccinate animals to reduce C. jejuni levels in said animals.

U.S. Pat. No. 5,773,000 teaches an immunoglobulin concentrate prepared from cows immunized against toxins A and B for treating C. difficile associated diseases such as colitis, pseudomembranous colitis and antibiotic associated diarrhea and especially for patients experiencing multiple relapses.

U.S. Pat. No. 5,529,910 teaches a PCR-based method for identifying causative bacteria of food poisoning in samples taken from infected patients.

U.S. Pat. No. 6,040,421 teaches the use of an adhesin from E. coli O157:H7 as either a competitive inhibitor or for immunization of cattle.

As discussed above, concerns about food and water quality are primary in many consumer's minds, so much so that there is a demand for products and means not only for simply treating diseases caused by enterotoxigenic organisms but proactive means for ensuring that contamination and colonization does not take place, thereby eliminating risks. Furthermore, there is considerable concern that over-use of antibiotics has led to the development of resistant bacterial strains, meaning that alternative approaches are clearly needed.

### SUMMARY OF THE INVENTION

According to a first aspect of the invention, there is provided a method of purging a localized environment of at least one enterotoxigenic organism comprising: providing an environment containing at least one enterotoxigenic organism; providing an antibody preparation, said preparation comprising antibodies against said organism; and exposing said environment to an effective amount of said antibody preparation sufficient to neutralize or purge said organism from said environment.

The enterotoxigenic organism may be selected from the group consisting of: Bacillus cereus, Bacillus anthracis, Bacillus subtilis, Bacillus thuringiensis, Bacillus stearothermophilus, Vibrio parahemolyticus, Vibrio cholerae O1, Vibrio cholerae non-O1, Vibrio vulnificus, Aeromonas hydrophilia, Salmonella enterica, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli (ETEC, EPEC, EHEC, EaggEC, UPEC and EIEC), Campylobacter jejuni, Campylobacter coli, Campylobacter lari, Campylobacter fetus, Yersinia enterocolitica, Yersinia pestis, Yersinia pseudotuberculosis, Listeria monocytogenes, Plesiomonas shigelloides, Shigella, Streptococcus, Giardia lamblia, Entamoeba histolytica, Cryptosporidium parvum, Cylcospora cayetanenis, Amisakis, Diphyllobothrium, Nanophyetus, Eustrongylides, Acanthamoeba, Ascaris lumbricoides, Trichuris trichuris, Hepatitis A virus, Hepatitis E virus, Rotavirus, Norwalk virus group, Acinetobacter, Actinobacillus, Actinomycetes, Actinomyces, Aeromonas, Peptostreptococcus, Veillonella, Mobiluncus, Propionibacterium acnes, Lactobacillus, Eubacterium, Bifidobacterium, Bacteroides, Prevotella, Porphyromonas, Fusobacterium, Bordetella, Borrelia, Brucella, Burkholderia, Citrobacter, Corynebacterium, Edwardsiella, Enterobacter, Enterobacteriaceae, Klebsiella, Morganella, Proteus, Providencia, Serratia, Enterococcus, Erysipelothrix rhusopathiae, Francisella tularensis, Haemophilus, Helicobacter, Legionella pneumophilia, Leptospira interrogans, Micrococcaceae, Moraxella catarrhalis, Mycobacterium, Nocardia, Neissaria, Pasteurella multocida, Pasteurellaceae, Pseudomonas aeruginosa, Rhodococcus, Serratia marcescens, Stenotrophomonas maltophilia, Streptococcus pneumoniae, Streptomyces, Treponema and combinations thereof.

The environment may be selected from the group consisting of: a gastrointestinal tract; a carcass; a birthing pen; a water supply; and a food product.

The antibody preparation may be in a powder form.

The antibody preparation may be a capsule, a liquid form or as an inhaler.

The organism may be a methanogen.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

As used herein, “animals” refers to vertebrates.

As used herein, “enterotoxigenic organism” refers to an organism capable of colonizing the gastrointestinal tract of an animal. Examples of enterotoxigenic microogranisms include but are by no means limited to Bacillus cereus, Bacillus anthracis, Bacillus subtilis, Bacillus thuringiensis, Bacillus stearothermophilus, Vibrio parahemolyticus, Vibrio cholerae O1, Vibrio cholerae non-O1, Vibrio vulnificus, Aeromonas hydrophilia, Salmonella enterica, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli (ETEC, EPEC, EHEC, EaggEC, UPEC and EIEC), Campylobacter jejuni, Campylobacter coli, Campylobacter lari, Campylobacter fetus, Yersinia enterocolitica, Yersinia pestis, Yersinia pseudotuberculosis, Listeria monocytogenes, Plesiomonas shigelloides, Shigella, Streptococcus, Giardia lamblia, Entamoeba histolytica, Cryptosporidium parvum, Cylcospora cayetanenis, Amisakis, Diphyllobothrium, Nanophyetus, Eustrongylides, Acanthamoeba, Ascaris lumbricoides, Trichuris trichuris, Hepatitis A virus, Hepatitis E virus, Rotavirus, Norwalk virus group, Acinetobacter, Actinobacillus, Actinomycetes, Actinomyces, Aeromonas, Peptostreptococcus, Veillonella, Mobiluncus, Propionibacterium acnes, Lactobacillus, Eubacterium, Bifidobacterium, Bacteroides, Prevotella, Porphyromonas, Fusobacterium, Bordetella, Borrelia, Brucella, Burkholderia, Citrobacter, Corynebacterium, Edwardsiella, Enterobacter, Enterobacteriaceae, Klebsiella, Morganella, Proteus, Providencia, Serratia, Enterococcus, Erysipelothrix rhusopathiae, Francisella tularensis, Haemophilus, Helicobacter, Legionella pneumophilia, Leptospira interrogans, Micrococcaceae, Moraxella catarrhalis, Mycobacterium, Nocardia, Neissaria, Pasteurella multocida, Pasteurellaceae, Pseudomonas aeruginosa, Rhodococcus, Serratia marcescens, Stenotrophomonas maltophilia, Streptococcus pneumoniae, Streptomyces, and Treponema.

As used herein, “passive immunity” refers to the transfer of antibodies from an immunized animal to a non-immune recipient.

As used herein, “effective amount” refers to a dosage sufficient to have the desired effect.

Enterotoxigenic microorganisms cause a number of diseases and disorders, including, for example, dysentery, gastroenteritis, typhoid fever, cholera, infectious hepatitis, poliomyelitis and diarrhea. Typically, normal motor propulsive activity of the GI tract limits the growth of organisms in the small intestine. Furthermore, the immune system is active within the GI tract, with B-cells and T-cells being located in several major compartments: for example, Peyer's patches, and in the lamina propria. Specifically, the lymphocytes in the lamina propria are thought to produce IgA. It is of note that IgA resists proteolysis but activates complement poorly. T cells mature in the thymus and both B-cells and T-cells migrate to the Peyer's patches. On stimulation by antigens or pathogens, these cells either become tolerant or activated, after which they migrate to mesenteric lymph nodes and then enter the systemic immune system to recirculate. Cells that have been stimulated in the mucosal immune system have a tendency to recirculate to the sites where they were stimulated (homing), repopulating the mucosal immune system with cells that have memory for gut antigens and that can mediate effector functions against pathogens on rechallenge. However, when the stress of excess pathogens and toxins is too overwhelming for the mucosal immune system, enterotoxigenic microorganisms colonize the GI tract, resulting in the diseases discussed above. As discussed above, in some cases, the enterotoxigenic microorganism is introduced into the host GI tract by ingestion of contaminated food or water.

As discussed above, there are several inventions relating to the use of antibodies to treat bacterial infections, for example, enterotoxigenic bacteria infections in animals as well as methods for using antibodies to detect harmful pathogens in meat or other end products. Thus, in one instance, the antibodies are used to treat infections in individuals afflicted with a specific disease whereas in the other, products are screened for the presence of harmful pathogens. Thus, the prior art teaches methods of treating diseases caused by enterotoxigenic organisms as well as methods for detecting the presence of said organisms when randomly screening food products so that further testing can occur or destruction of the food products can take place.

However, the prior art does not teach methods of neutralizing or purging the microorganisms from a localized environment so that contamination or infection does not occur. Specifically, animals often develop a tolerance to the infecting enterotoxigenic micro-organism and do not develop any symptoms from these infections, or the microorganism may inhabit the gastrointestinal tract of the animal without harming the animal but may still have undesirable effects on the environment, such as methanogenic bacteria, as discussed below. As such, administering antibodies to these animals is not taught by the prior art as this does not constitute treating the infection, as there are no symptoms. Furthermore, the prior art in fact teaches against administering antibodies to animals not showing symptoms as the prior art predicts that this would cause the micro-organisms to be flushed from the GI tract into the local environment where the micro-organisms could infect other, non-tolerant animals.

The prior art teaches that colostral or egg yolk antibodies present in the GI tract can combine with disease-causing microorganisms to reduce their movement and adhesive properties. This in turn allows the disease-causing microorganisms to be more easily flushed from the GI tract.

However, the prior art does not teach the use of antibodies to inhibit growth of enterotoxigenic microorganisms in environments other than the GI tract.

Specifically, herein described is a method of treating a localized environment with a sufficient amount of an antibody preparation to neutralize the enterotoxigenic micro-organisms in the localized environment such that subsequent contamination or infection does not occur. In addition, it is herein shown that antibodies can be used to inhibit bacterial growth independent of a host immune system.

Also described is a method of inhibiting growth of microorganisms within a localized environment comprising administering antibodies as discussed below into a localized environment, thereby inhibiting growth of the micro-organisms, wherein the antibodies are directed against one or more specific micro-organisms known or suspected of inhabiting or contaminating the localized environment.

As a consequence, the antibodies can be used for treating a number of different environments not previously considered for antibody treatment, as well as in a number of different products. For example, the antibodies can be used to treat: water supplies suspected of enterotoxigenic microorganism contamination; surfaces at risk of contamination, for example, counter tops, meat processing areas; or food products. As will be appreciated by one knowledgeable in the art, in these embodiments, the antibodies are acting effectively as preservatives in that addition of the antibodies to the localized environment inhibits bacterial growth within the environment, thereby greatly reducing the risk of subsequent infection (because of reduced bacterial levels) and GI tract colonization (because of growth inhibition).

In one embodiment, the antibodies are obtained from eggs of immunized hens. As will be appreciated by one knowledgeable in the art, other fowl may be used as may other sources of antibodies known in the art.

Thus, in some embodiments, hens are immunized against an antigen from a enterotoxigenic organism or the organism itself. Eggs are then collected and the antibodies may be purified or concentrated using means known in the art.

In some embodiments, the antibodies may be antibodies from hens immunized against two or more antigens from one enterotoxigenic organism or from two or more enterotoxigenic organisms or may be a mixture of antibodies from hens immunized against different antigens and/or organisms. As will be appreciated by one knowledgeable in the art, the combinations may be selected for treating a specific animal, for example, antibodies against Salmonella and Campylobacter for use in chickens, a specific type of disorder, for example, diarrhea, or may be a combination of antibodies against common pathogens or pathogens of a particular animal.

The antibodies may be employed alone as a liquid or solid, preferably in a solid powder form and preferably in admixture with a carrier to form a pharmaceutical composition such as a tablet, capsule or suppository. In other embodiments, discussed below, the antibodies are in a powder form and may be used with, for example, a spray applicator or an inhaler. In yet other embodiments, the antibodies may be added to or incorporated into food products, for example, condiments and seasonings, as discussed below.

Since preferred methods of administration are oral and rectal, or enteric installation, tablets and capsules are preferred. These of course are prepared according to conventional methods known in the art. The antibodies, as discussed below, may be combined with other pharmaceutically acceptable carriers such as various liquids, proteins or oils which may also provide additional nutritional and/or pharmaceutical benefits.

In some embodiments, the antibodies discussed above may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling and arranged for sustained release or a quick burst. In some embodiments, carriers of different classes may be used in combination for multiple effects, for example, a quick burst followed by sustained release.

The invention provides kits for carrying out the methods of the invention. Accordingly, a variety of kits are provided.

The kits of the invention comprise one or more containers comprising antibodies, a suitable excipient as described herein and a set of instructions, generally written instructions although electronic storage media (e.g., magnetic diskette or optical disk) containing instructions are also acceptable, relating to the use and dosage of the antibodies.

The antibodies of the kit may be packaged in any convenient, appropriate packaging. For example, if the antibodies are a freeze-dried formulation, an ampoule with a resilient stopper is normally used, so that the drug may be easily reconstituted by injecting fluid through the resilient stopper. Also, prefilled syringes may be used when the kit is supplied with a liquid formulation of the antibodies.

In other embodiments, the antibodies are incorporated into other food products, for example, condiments or seasonings, for use with meats and the like. As discussed above, in these embodiments, the antibodies are acting effectively as a preservative. In yet other embodiments, the antibodies are a powder for adding to food products or for dusting or treating a localized environment suspected of contamination by one or more micro-organisms.

The invention will now be described by way of examples. However, the invention is not limited to the examples.

### EXAMPLE I

### Inhibition of Growth with Antibodies

Chicken egg yolk antibodies were produced against the outer membrane proteins and the lipopolysaccharides from S. typhemerium and S. enteritis. Preliminary studies demonstrated that the antibodies when used alone in an in vitro incubation system substantially inhibited the growth of the corresponding organisms. These data support the proposal that the specific antibodies can be used singly or in combination to inhibit growth of the serovars of Salmonella in food products. These surprising results indicate that antibodies against other food-borne pathogens would inhibit growth of those pathogens as well.

### EXAMPLE II

### Removal of Enterotoxigenic Organisms from a Birthing Pen

Colibacillosis costs the hog and cattle industries millions of dollars each year. Specifically, enterotoxigenic organisms, such as strains of E. coli, are shed with fecal material by, for example, sows and cows. It is of note that these animals are resistant to the enterotoxigenic organisms and therefore do not develop any symptoms of the associated diseases. However, newborn piglets and calves are immunodeficient, relying on antibodies passively transferred in the milk from their mother. It is of note that under certain circumstances, the milk may be devoid or deficient in certain antibodies, for example, anti-E. coli antibodies. As a consequence, when the newborns come in contact with the enterotoxigenic organisms present in the fecal matter, they are infected by the enterotoxigenic organism and develop symptoms of the disease, for example, colibacillosis. U.S. Pat. No. 5,080,895, discussed above, teaches treating or preventing development of the disease in newborns by administering an antibody-containing substance.

However, an alternative method not considered is to treat the sows and cows prior to birthing with an antibody preparation as described above. Specifically, the antibody preparation is taken up by the adult animal and the antibodies purge the enterotoxigenic organism from the gastrointestinal tract of the adult animals and furthermore inhibit growth of the micro-organisms. As a result, greatly reduced levels of enterotoxigenic organisms are subsequently shed in the fecal matter, meaning that there are reduced levels of pathogen to contaminate the newborns. It is of note that in these embodiments, the antibodies may be fed to the animals as a powder or may be incorporated into feed.

### EXAMPLE III

### Purging of Enterotoxigenic Organisms from Livestock Prior to Slaughter

As discussed above, contamination of food products with enterotoxigenic bacteria often occurs when a tainted product, for example, meat, comes in contact with other food products. The problem is that enterotoxigenic organisms which do not cause disease in livestock remain in the intestine of the animal during slaughter. As a consequence, the organisms can contaminate end products during processing or other products during handling. There are several examples in the prior art of methods and kits for screening random samples of a food product for the presence of enterotoxigenic bacteria.

However, rather than relying on chance to detect tainted products, in these embodiments, livestock are fed antibody preparations as described above a suitable time period before slaughter. As will be appreciated by one knowledgeable in the art, this time period is long enough that substantially all of the enterotoxigenic organism is purged from the gastrointestinal tract of the animal but not so long without treatment that recolonization can occur. In some embodiments, treatment may be continuous. As a consequence, when the animal is slaughtered, there are no pathogenic organisms to contaminate other food products.

In some embodiments, the purging of the gastrointestinal tract via passive immunization may be combined with other treatments known in the art, for example, vaccination and/or antibiotics if desired so that the level of enterotoxigenic organisms is reduced prior to purging.

### EXAMPLE IV

### Treatment of Surface with Antibody Powder

As discussed above, major sources of enterotoxigenic microorganism contamination are areas where food is handled, for example, slaughter houses, meat processing plants, restaurant and kitchen counter tops, produce handling centres and the like. That is, during handling and/or packaging, microorganisms from a single source can be transmitted to several products, thereby spreading contamination. These microorganisms in turn grow and divide, thereby increasing the likelihood of further contamination. Furthermore, as will be apparent, the greater the quantity of enterotoxigenic microorganisms ingested, the more likely it is that the microorganisms will overwhelm the GI tract immune system. As discussed above, the prior art teaches the use of antibodies to treat the disorders caused by microorganisms once symptoms have developed; however, the prior art does not teach applying antibody powder or spray to surfaces suspected of enterotoxigenic microorganism contamination. As discussed above, applying the antibodies to these surfaces will inhibit growth of any microorganisms present, thereby reducing the risk of further contamination or subsequent illness. In these embodiments, the antibodies may be applied as, for example, a powder, spray or liquid solution as well as in other suitable forms known in the art.

### EXAMPLE V

### Prevention of Environmental Contamination

As discussed above, there are considerable concerns regarding contamination of ground water and other water supplies with fecal material from livestock containing enterotoxigenic organisms. As will be appreciated by one knowledgeable in the art, these organisms may or may not be harmful to their original host but may cause disease following infection and colonization of, for example, human GI tracts. In these embodiments, antibodies as described above are fed to the animal, which inhibits growth of the enterotoxigenic organism within the animal's GI tract. As will be apparent to one knowledgeable in the art, whether the antibodies cause the organism to be purged in addition to inhibiting growth of the organism, the amount of enterotoxigenic organism shed will be greatly reduced. This in turn will reduce the chance of secondary infections of other animals and also greatly reduce the risk of contamination of water supplies.

As will be appreciated by one knowledgeable in the art, the antibodies may be used as a preservative or rather as an anti-microorganism agent which is added to food or liquid at risk of microorganism contamination. For example, the antibodies described above may be mixed in seasonings or condiments used with meat products or may be added to water supplies or reservoirs.

### EXAMPLE VI

### Reduction of Methane Gas Production

Methane is a potent greenhouse gas, some twenty-one times more potent than carbon dioxide. It has been estimated that approximately 15% of methane gas emissions world-wide come from livestock (McAllister et al., 1996, Can J Anim Sci 76: 231-243). The methane gas is produced by micro-organisms which inhabit a ruminant's stomach and/or GI tract and have been termed mathanogens. Examples of mathanogenic organisms include but are by no means limited toMethanobrevibacter ruminantium, Methanobrevibacter sp., Methanosarcina barkeri, Methanosarcina mazei, Methanobacterium formicium and Methanomicrobium mobile. Treating or administering the ruminants with an antibody preparation as described above, that is, containing antibodies directed against one or more known methanogens, would result in reduction of the number of methanogens within the ruminant GI tract, thereby reducing the methane produced. Specifically, the antibody preparation is taken up by the ruminant and the antibodies purge the methanogen from the gastrointestinal tract of the animal.

While the preferred embodiments of the invention have been described above, it will be recognized and understood that various modifications may be made therein, and the appended claims are intended to cover all such modifications which may fall within the spirit and scope of the invention.

1. A method of purging a localized environment of at least one enterotoxigenic organism comprising:
providing an environment containing at least one enterotoxigenic organism;
providing an antibody preparation, said preparation comprising antibodies against said organism; and
exposing said environment to an effective amount of said antibody preparation sufficient to neutralize or purge said organism from said environment.

2. The method according to claim 1 wherein the enterotoxigenic organism is selected from the group consisting of: Bacillus cereus, Bacillus anthracis, Bacillus subtilis, Bacillus thuringiensis, Bacillus stearothermophilus, Vibrio parahemolyticus, Vibrio cholerae O1, Vibrio cholerae non-O1, Vibrio vulnificus, Aeromonas hydrophilia, Salmonella enterica, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli (ETEC, EPEC, EHEC, EaggEC, UPEC and EIEC), Campylobacter jejuni, Campylobacter coli, Campylobacter lari, Campylobacter fetus, Yersinia enterocolitica, Yersinia pestis, Yersinia pseudotuberculosis, Listeria monocytogenes, Plesiomonas shigelloides, Shigella, Streptococcus, Giardia lamblia, Entamoeba histolytica, Cryptosporidium parvum, Cylcospora cayetanenis, Amisakis, Diphyllobothrium, Nanophyetus, Eustrongylides, Acanthamoeba, Ascaris lumbricoides, Trichuris trichuris, Hepatitis A virus, Hepatitis E virus, Rotavirus, Norwalk virus group, Acinetobacter, Actinobacillus, Actinomycetes, Actinomyces, Aeromonas, Peptostreptococcus, Veillonella, Mobiluncus, Propionibacterium acnes, Lactobacillus, Eubacterium, Bifidobacterium, Bacteroides, Prevotella, Porphyromonas, Fusobacterium, Bordetella, Borrelia, Brucella, Burkholderia, Citrobacter, Corynebacterium, Edwardsiella, Enterobacter, Enterobacteriaceae, Klebsiella, Morganella, Proteus, Providencia, Serratia, Enterococcus, Erysipelothrix rhusopathiae, Francisella tularensis, Haemophilus, Helicobacter, Legionella pneumophilia, Leptospira interrogans, Micrococcaceae, Moraxella catarrhalis, Mycobacterium, Nocardia, Neissaria, Pasteurella multocida, Pasteurellaceae, Pseudomonas aeruginosa, Rhodococcus, Serratia marcescens, Stenotrophomonas maltophilia, Streptococcus pneumoniae, Streptomyces, Treponema and combinations thereof.

3. The method according to claim 1 wherein the environment is selected from the group consisting of: a gastrointestinal tract; a carcass; a birthing pen; a water supply; and a food product.

4. The method according to claim 1 wherein the antibody preparation is in a powder form.

5. The method according to claim 1 wherein the antibody is a capsule.

6. The method according to claim 1 wherein the antibody is in a liquid form.

7. The method according to claim 1 wherein the antibody is in a spray.

8. The method according to claim 1 wherein the antibody preparation is derived from eggs.

9. The method according to claim 1 wherein the organism is a methanogen.
